(19) United States
(12) Patent Application Publication
(10) Pub. No.: US 2024/0051964 A1
(43) Pub. Date: Feb. 15, 2024

(54) SOLID STATE FORMS OF CAPIVASERTIB AND PROCESS FOR PREPARATION THEREOF

(71) Applicant: TEVA PHARMACEUTICALS INTERNATIONAL GMBH, Jona (CH)

(72) Inventors: Valentina Travancic, Zagreb (HR); Dijana Skalec Samec, Jastrebarsko (HR)

(21) Appl. No.: 18/265,724

(22) PCT Filed: Jan. 4, 2022
(86) PCT No.: PCT/US2022/011073

§ 371 Date: Jun. 7, 2023

Related U.S. Application Data
(60) Provisional application No. 63/139,342, filed on Jan. 20, 2021.
(60) Provisional application No. 63/133,529, filed on Jan. 4, 2021.

(51) Int. Cl. C07D 487/04

(52) U.S. Cl. CPC C07D 487/04

(57) ABSTRACT

The present disclosure encompasses solid state forms of Capivasertib, in embodiments crystalline polymorphs, salts and co-crystals of Capivasertib, processes for preparation thereof, and pharmaceutical compositions thereof.

### FIELD OF THE DISCLOSURE

The present disclosure encompasses solid state forms of Capivasertib, in embodiments crystalline polymorphs, salts and co-crystals of Capivasertib, processes for preparation thereof, and pharmaceutical compositions thereof.

### BACKGROUND OF THE DISCLOSURE

Capivasertib, 4-amino-N-[(15)-1-(4-chlorophenyl)-3-hydroxypropyl]-1-(7H-pyrrolo[2,3-d]pyrimidin-4-yl)piperidine-4-carboxamide, has the following chemical structure:

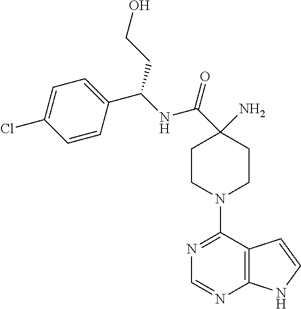 [SMILES: NC1(C(=O)N[C@@H](CCO)c2ccc(Cl)cc2)CCN(c2ncnc3[nH]ccc23)CC1]

Capivasertib is an orally available inhibitor of the serine/threonine protein kinase AKT with potential antineoplastic activity. It has been investigated for the treatment of cancer; in particular metastatic breast cancer.

The compound and salts thereof, are described in U.S. Pat. No. 8,101,623.

U.S. Pat. No. 9,487,525 describes solid forms A, B and C of Capivasertib.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including co-crystals) of Capivasertib.

### SUMMARY OF THE DISCLOSURE

The present disclosure encompasses solid state forms of Capivasertib, in embodiments crystalline polymorphs, salts and co-crystals of Capivasertib, processes for preparation thereof, and pharmaceutical compositions thereof. These crystalline polymorphs, salts and co-crystals can be used to prepare other solid state forms of Capivasertib, Capivasertib salts, co-crystals and their solid state forms.

The present disclosure also provides uses of the said solid state forms of Capivasertib, salts and co-crystals in the preparation of other solid state forms of Capivasertib or salts or co-crystals thereof.

The present disclosure provides crystalline polymorphs, salts and co-crystals of Capivasertib for use in medicine, including for the treatment of cancer.

The present disclosure also encompasses the use of crystalline polymorphs, salts and co-crystals of Capivasertib of the present disclosure for the preparation of pharmaceutical compositions and/or formulations.

In another aspect, the present disclosure provides pharmaceutical compositions comprising crystalline polymorphs, salts and co-crystals of Capivasertib according to the present disclosure.

The present disclosure includes processes for preparing the above mentioned pharmaceutical compositions. The processes include combining any one or a combination of the crystalline polymorphs of Capivasertib, salts and co-crystals with at least one pharmaceutically acceptable excipient.

The crystalline polymorph of Capivasertib, salts and co-crystals as defined herein and the pharmaceutical compositions or formulations of the crystalline polymorph, salts and co-crystals of Capivasertib may be used as medicaments, such as for the treatment of cancer; in particular metastatic breast cancer.

The present disclosure also provides methods of treating cancer, such metastatic breast cancer, by administering a therapeutically effective amount of any one or a combination of the crystalline polymorphs, salts and co-crystals of Capivasertib of the present disclosure, or at least one of the above pharmaceutical compositions, to a subject suffering from cancer, or otherwise in need of the treatment.

The present disclosure also provides uses of crystalline polymorphs, salts and co-crystals of Capivasertib of the present disclosure, or at least one of the above pharmaceutical compositions, for the manufacture of medicaments for treating e.g. cancer, such as breast cancer.

### BRIEF DESCRIPTION OF THE DRAWINGS

FIG. 1 shows a characteristic X-ray powder diffraction pattern (XRPD) of crystalline Capivasertib:methyl paraben—Form 1.

FIG. 2 shows a characteristic XRPD of crystalline Capivasertib:glutaric acid—Form 2.

FIG. 3 shows a characteristic XRPD of crystalline Capivasertib: propyl paraben—Form 3.

FIG. 4 shows a characteristic XRPD of crystalline Capivasertib hydrochloride salt—Form H1.

FIG. 5 shows a characteristic XRPD of crystalline Capivasertib dihydrochloride salt—Form H2.

FIG. 6 shows a characteristic XRPD of amorphous Capivasertib.

FIG. 7 shows a characteristic XRPD of crystalline Capivasertib:sebacic acid—Form 4.

FIG. 8 shows a characteristic XRPD of crystalline Capivasertib—Form A.

FIG. 9 shows a characteristic XRPD of crystalline Capivasertib—Form B.

FIG. 10 shows characteristic XRPD pattern of Capivasertib—Form A corresponding to FIG. 1 of U.S. Pat. No. 9,487,525

FIG. 11 shows characteristic XRPD pattern of Capivasertib—Form B corresponding to FIG. 3.1 of U.S. Pat. No. 9,487,525

FIG. 12 shows characteristic XRPD pattern of Capivasertib—Form B corresponding to FIG. 3.2 of U.S. Pat. No. 9,487,525.

### DETAILED DESCRIPTION OF THE DISCLOSURE

The present disclosure encompasses solid state forms of Capivasertib, including crystalline polymorphs of Capivasertib, processes for preparation thereof, and pharmaceutical compositions thereof.

Solid state properties of Capivasertib and crystalline polymorphs thereof, as well as salts and co-crystals can be influenced by controlling the conditions under which Capivasertib and crystalline polymorphs, salts and co-crystals thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Capivasertib, or a Capivasertib salt or a co-crystal described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Capivasertib, or a Capivasertib salt or a co-crystal. In some embodiments of the disclosure, the described crystalline polymorph of Capivasertib, or a Capivasertib salt or a co-crystal may contain from about 1% to about 20% (w/w), from about 5% to about 20% (w/w), or from about 5% to about 10% (w/w) of one or more other crystalline polymorph of the same or a Capivasertib salt or a co-crystal.

Depending on which other crystalline polymorphs a comparison is made, the crystalline polymorphs of Capivasertib, or a Capivasertib salt or a co-crystal of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Capivasertib, or a Capivasertib salt or a co-crystal referred to herein as being characterized by graphical data “as depicted in” or “as substantially depicted in” a Figure will thus be understood to include any crystal forms of Capivasertib, or a Capivasertib salt or a co-crystal characterized with the graphical data having such small variations, as are well known to the skilled person, in comparison with the Figure.

As used herein, and unless stated otherwise, the term “anhydrous” in relation to crystalline forms of Capivasertib, or a Capivasertib salt or a co-crystal relates to a crystalline form of Capivasertib, salt or co-crystal which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

“Co-Crystal” or “Co-crystal” as used herein is defined as a crystalline material including two or more molecules in the same crystalline lattice which associated by non-ionic and non-covalent bonds. In some embodiments, the co-crystal includes two molecules which are in natural state. In an embodiments the molar ratio between the active pharmaceutical ingredient (Capivasertib) and the coformer (i.e. methyl paraben, glutaric acid or propyl paraben) is between 2:1 and 1:2, in embodiments 1:1.5 and 1.5:1, in some embodiments between 1:1.25 and 1.25:1, in other embodiments about 1:1.

As used herein, the term “isolated” in reference to crystalline polymorph of Capivasertib or a Capivasertib salt or a co-crystal of the present disclosure corresponds to a crystalline polymorph of Capivasertib or a Capivasertib salt or a co-crystal that is physically separated from the reaction mixture in which it is formed.

As used herein, unless stated otherwise, the XRPD measurements are taken using copper Kα radiation wavelength 1.5418 Å. XRPD peaks reported herein are measured using CuKα radiation, λ=1.5418 Å, typically at a temperature of 25±3° C.

As used herein, unless stated otherwise, ¹³C NMR reported herein are measured at 125 MHz at a magic angle spinning frequency φr/27c=11 kHz, preferably at a temperature of at 293 K±3° C.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

As used herein, Form A and Form B of Capivasertib is as defined in U.S. Pat. No. 9,487,525.

Form A of Capivasertib may be defined as having an powder X-ray diffraction pattern (measured using CuKα radiation having a wavelength of 1.54 Å) having characteristic peaks at: 3.1, 9.2, 14.3, 15.5, 16.5, 17.4, 18.9, 19.6, 23.5, and 25.9 degrees 2-theta±0.2 degrees 2-theta, optionally with the following corresponding relative intensity values (%): 39.7, 17.8, 100, 40.8, 28.2, 32.2, 64.9, 67.2, 55.2, and 49.4, respectively. Form A of Capivasertib may also be defined as having a powder X-ray diffraction pattern substantially as depicted in FIG. 10.

Alternatively Form A may be defined as having a powder X-ray diffraction pattern having characteristic peaks at 9.2, 14.2, 15.5, 17.3 and 18.8 degrees 2-theta±0.2 degrees 2-theta, and optionally having one, two, three, four or five additional peaks at 15.8, 17.9, 195, 21.5 and 25.8 degrees 2-theta±0.2 degrees 2-theta. Form A of Capivasertib may also be defined as having a powder X-ray diffraction pattern substantially as depicted in FIG. 8.

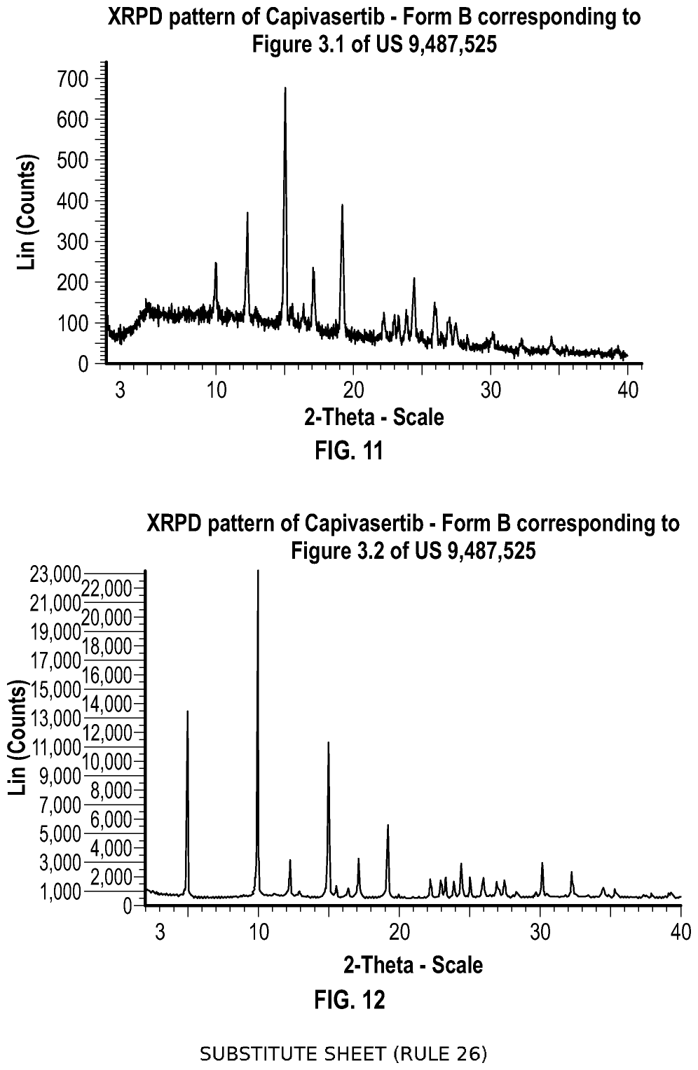

Form B of Capivasertib may be defined as having an powder X-ray diffraction pattern (measured using CuKα radiation having a wavelength of 1.54 Å) having characteristic peaks at 12.3, 15.0 and 19.2 degrees 2-theta±0.2 degrees 2-theta; or at 10.0, 12.3, 15.0, 17.1, 19.2 and 24.4 degrees 2-theta±0.2 degrees 2-theta; or at 15.0, 19.2, 12.3, 10.0, 17.1, 24.4, 16.4, 26.0, 15.5 and 23.9 degrees 2-theta±0.2 degrees 2-theta; or at 10.0, 5.0, 15.0, 19.2, 17.1, 12.3, 24.4, 30.2, 32.2 and 23.3 degrees 2-theta±0.2 degrees 2-theta. Alternatively, Form B of Capivasertib may be defined as having an powder X-ray diffraction pattern (measured using CuKα radiation having a wavelength of 1.54 Å) having characteristic peaks at: 10.0, 12.3, 15.0, 15.5, 16.4, 17.1, 19.2, 23.9, 24.4, and 26.0 degrees 2-theta±0.2 degrees 2-theta, optionally with the following corresponding relative intensity values (%): 36.0, 54.7, 100, 21.2, 22.1, 32.7, 57.7, 19.0, 31.0, and 21.8, respectively. Form B of Capivasertib may be alternatively defined as having an powder X-ray diffraction pattern (measured using CuKα radiation having a wavelength of 1.54 Å) having characteristic peaks at: 5.0, 10.0, 12.3, 15.0, 17.1, 19.2, 23.3, 24.4, 30.2, and 32.3 degrees 2-theta±0.2 degrees 2-theta, optionally with the following corresponding relative intensity values (%): 57.7, 100, 13.8, 48.7, 14.1, 24, 8.2, 12.4, 11.4, and 9.8, Form B of Capivasertib may also be defined as having a powder X-ray diffraction pattern substantially as depicted in FIG. 11 or FIG. 12.

Alternatively Form B may be defined as having a powder X-ray diffraction pattern having characteristic peaks at 12.3, 15.0, 17.1, 19.2, and 24.4 degrees 2-theta±0.2 degrees 2-theta, and optionally having one, two, three, four or five additional peaks at 9.9, 16.4, 22.2, 23.9, and 26.0 degrees 2-theta±0.2 degrees 2-theta. Form B of Capivasertib may also be defined as having a powder X-ray diffraction pattern substantially as depicted in FIG. 8.

In one aspect, the disclosure provides a co-crystal of Capivasertib with an alkyl ester of para-hydroxybenzoic acid (i.e. an alkyl paraben). The co-crystal of Capivasertib may be a C₁ to C₆ alkyl ester of para-hydroxybenzoic acid, and particularly a C₁ to C₃ alkyl ester of para-hydroxybenzoic acid.

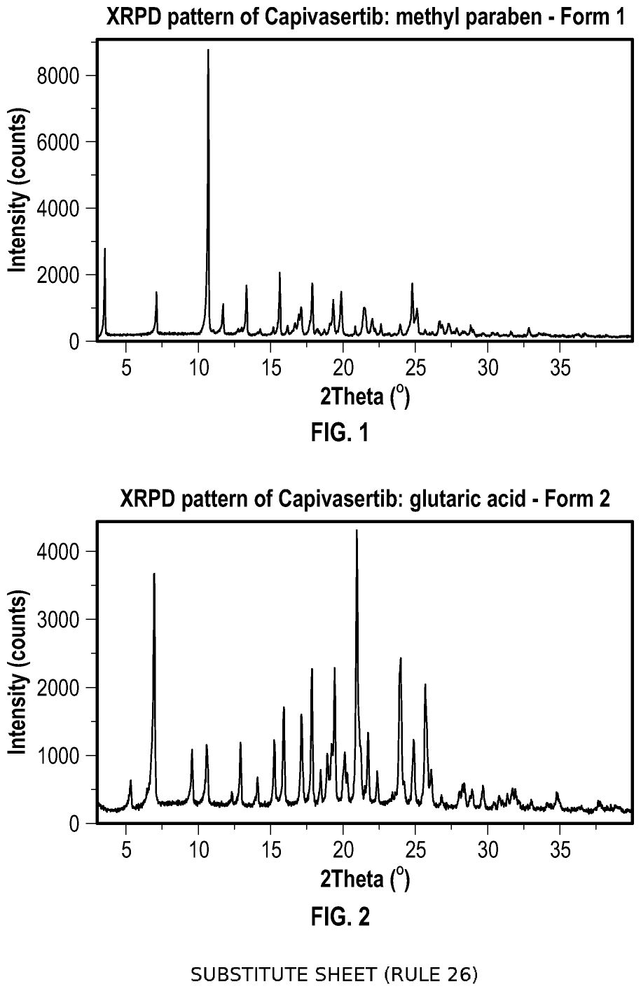

The disclosure encompasses a crystalline form of Capivasertib:methyl paraben, designated Form 1. Form 1 of Capivasertib:methyl paraben, may be characterized by data selected from one or more of the following: an X-ray powder diffraction pattern substantially as depicted in FIG. 1; an X-ray powder diffraction pattern having peaks at 3.6, 7.2, 10.8, 11.7 and 13.4 degrees 2-theta±0.2 degrees 2-theta; and combinations of these data.

Form 1 of Capivasertib:methyl paraben may be further characterized by an X-ray powder diffraction pattern having peaks at 3.6, 7.2, 10.8, 11.7 and 13.4 degrees 2-theta±0.2 degrees 2-theta, and also having any one, two, three or four additional peaks selected from 15.7, 17.9, 19.3 and 19.9 degrees 2-theta±0.2 degrees 2-theta.

Form 1 of Capivasertib:methyl paraben may be further characterized by an X-ray powder diffraction pattern having peaks at 3.6, 7.2, 10.8, 11.7, 13.4, 17.9, 19.3 and 19.9 degrees 2-theta±0.2 degrees 2-theta.

Form 1 of Capivasertib:methyl paraben may be further characterized by an X-ray powder diffraction pattern having peaks at 3.6, 7.2, 10.8, 11.7, 13.4, 15.7, 17.9, 19.3, and 19.9 degrees 2-theta±0.2 degrees 2-theta.

In embodiments of the present disclosure, crystalline Form 1 of Capivasertib:methyl paraben is isolated.

In another embodiment, Crystalline Form 1 of Capivasertib:methyl paraben may be a co-crystal of Capivasertib and methyl paraben.

In any embodiment, the ratio of Capivasertib to methyl paraben in Form 1 of Capivasertib:methyl paraben, is 1:2 to 2:1, or 1:1.5 to 1.5:1, or 1.2:1 to 1:1.2, or 1:1.1 or 1.1:1, or about 1:1.

According to any aspect or embodiment, crystalline Capivasertib:alkyl paraben, particularly crystalline Capivasertib:(C₁-C₆ alkyl) paraben, or crystalline Capivasertib:(C₁-C₃ alkyl) paraben, or crystalline Capivasertib:methyl paraben, may be polymorphically pure. Additionally, or alternatively, the crystalline Capivasertib:methyl paraben, may be enantiomerically pure.

The disclosure further encompasses a crystalline form of Capivasertib:glutaric acid, designated Form 2. Form 2 of Capivasertib:glutaric acid may be characterized by data selected from one or more of the following: an X-ray powder diffraction pattern substantially as depicted in FIG. 2; an X-ray powder diffraction pattern having peaks at 7.0, 10.6, 15.9, 17.9 and 20.9 degrees 2-theta±0.2 degrees 2-theta; and combinations of these data.

Form 2 of Capivasertib:glutaric acid may be further characterized by an X-ray powder diffraction pattern having peaks at 7.0, 10.6, 15.9, 17.9 and 20.9 degrees 2-theta±0.2 degrees 2-theta, and also having any one, two, three, four or five additional peaks selected from 5.3, 9.6, 15.3, 18.4 and 24.9 degrees 2-theta±0.2 degrees 2-theta.

Form 2 of Capivasertib:glutaric acid may be further characterized by an X-ray powder diffraction pattern having peaks at 5.3, 7.0, 9.6, 10.6, 15.3, 15.9, 17.9, 18.4, 20.9 and 24.9 degrees 2-theta±0.2 degrees 2-theta.

In embodiments of the present disclosure, crystalline Form 2 of Capivasertib:glutaric acid is isolated.

In any embodiment, the ratio of Capivasertib to glutaric acid in Form 2 of Capivasertib:glutaric acid, is 1:2 to 2:1, or 1:1.5 to 1.5:1, or 1.2:1 to 1:1.2, or 1:1.1 or 1.1:1, or about 1:1. Particularly, the ratio Capivasertib:glutaric acid is 1:1.

In one embodiment, crystalline Form 2 of Capivasertib:glutaric acid may be a co-crystal of Capivasertib and glutaric acid.

In another embodiment, crystalline Form 2 of Capivasertib:glutaric acid may be a salt.

According to any embodiment, the crystalline Capivasertib:glutaric acid may be polymorphically pure. Additionally, or alternatively, the crystalline Capivasertib:glutaric acid, may be enantiomerically pure.

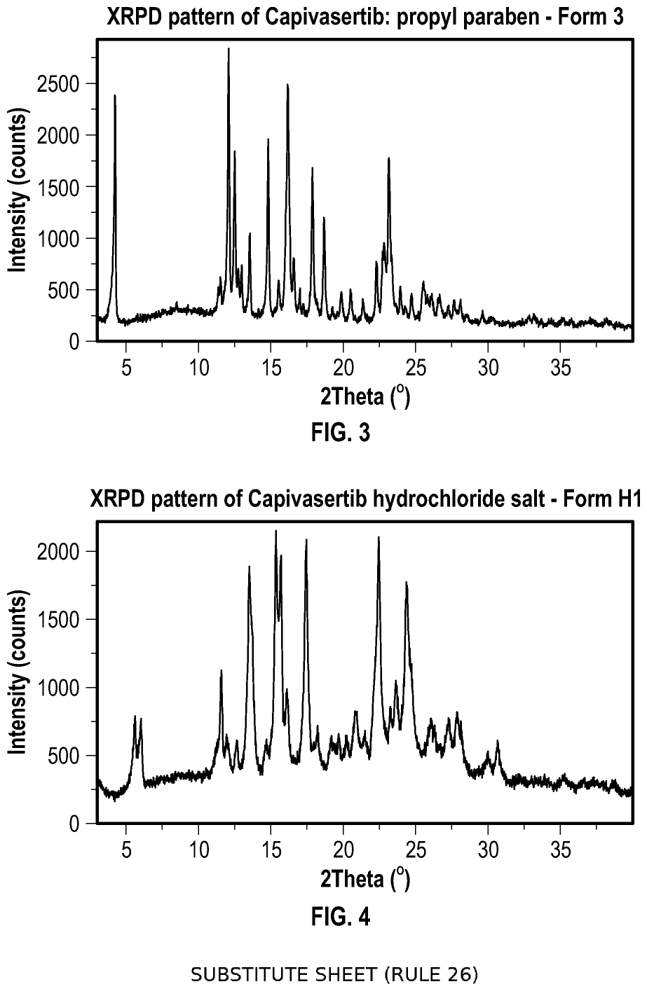

In another embodiment, the present disclosure includes a crystalline form of Capivasertib: propyl paraben, designated Form 3. Form 3 of Capivasertib: propyl paraben may be characterized by data selected from one or more of the following: an X-ray powder diffraction pattern substantially as depicted in FIG. 3; an X-ray powder diffraction pattern having peaks at 4.3, 13.5, 16.2, 17.9 and 18.7 degrees 2-theta±0.2 degrees 2-theta; and combinations of these data.

Form 3 of Capivasertib: propyl paraben may be further characterized by an X-ray powder diffraction pattern having peaks at 4.3, 13.5, 16.2, 17.9 and 18.7 degrees 2-theta±0.2 degrees 2-theta, and also having any one, two, three or four additional peaks selected from 12.5, 20.5, 22.3 and 23.1 degrees 2-theta±0.2 degrees 2-theta.

Crystalline Form 3 of Capivasertib: propyl paraben may be further characterized by an X-ray powder diffraction pattern having peaks at 4.3, 12.5, 13.5, 16.2, 17.9, 18.7, 20.5, 22.3 and 23.1 degrees 2-theta±0.2 degrees 2-theta.

In embodiments of the present disclosure, crystalline Form 3 of Capivasertib: propyl paraben is isolated.

In another embodiment, Crystalline Form 3 of Capivasertib: propyl paraben may be a co-crystal of Capivasertib and propyl paraben.

According to any aspect or embodiment, crystalline Capivasertib: propyl paraben may be polymorphically pure. Additionally, or alternatively, the crystalline Capivasertib: propyl paraben may be enantiomerically pure.

The present disclosure includes also crystalline Capivasertib hydrochloride salt; designated Form H1. Form H1 of Capivasertib hydrochloride salt may be characterized by data selected from one or more of the following: an X-ray powder diffraction pattern substantially as depicted in FIG. 4; an X-ray powder diffraction pattern having peaks at 11.6, 13.5, 17.4, 22.5 and 23.7 degrees 2-theta±0.2 degrees 2-theta; and combinations of these data.

From H1 of Capivasertib hydrochloride salt may be further characterized by an X-ray powder diffraction pattern having peaks at 11.6, 13.5, 17.4, 22.5 and 23.7 degrees 2-theta±0.2 degrees 2-theta, and also having any one, two, three, four or five additional peaks selected from 12.7, 15.3, 16.1, 18.2 and 27.8 degrees 2-theta±0.2 degrees 2-theta.

Crystalline Capivasertib hydrochloride salt form H1 may be further characterized by an X-ray powder diffraction pattern having peaks at 11.6, 12.7, 13.5, 15.3, 16.1, 17.4, 18.2, 22.5, 23.7 and 27.8 degrees 2-theta±0.2 degrees 2-theta.

According to any aspect or embodiment, crystalline Capivasertib hydrochloride salt form H1 may be polymorphically pure. Additionally, or alternatively, the crystalline Capivasertib hydrochloride salt form H1 may be enantiomerically pure.

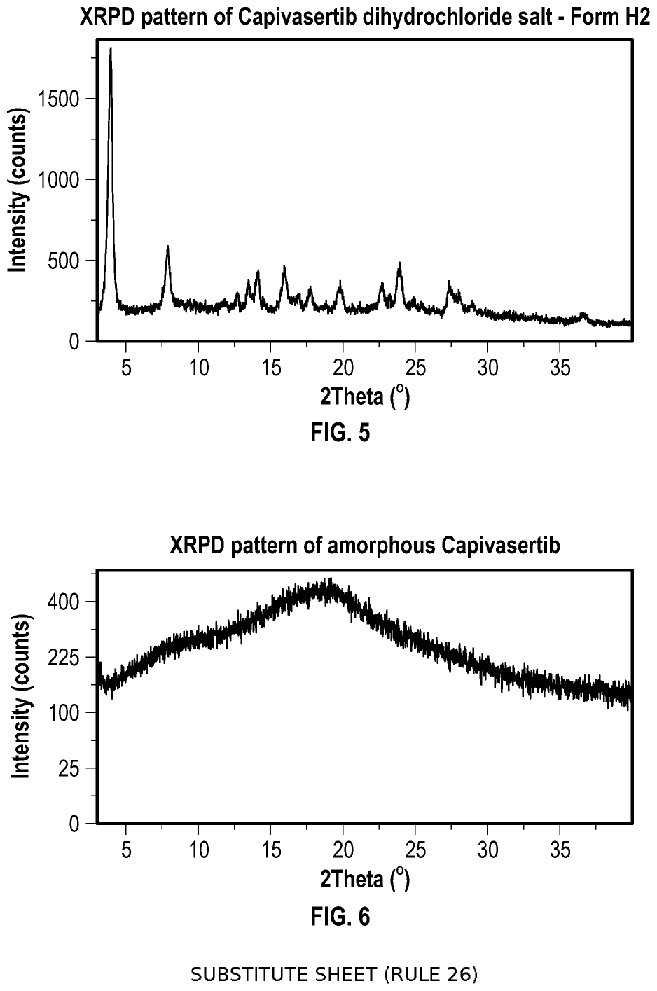

In a further embodiment, the present disclosure encompasses crystalline Capivasertib dihydrochloride salt; designated Form H2. Form H2 of Capivasertib dihydrochloride salt may be characterized by data selected from one or more of the following: an X-ray powder diffraction pattern substantially as depicted in FIG. 5; an X-ray powder diffraction pattern having peaks at 3.9, 7.9, 14.2, 16.0 and 23.8 degrees 2-theta±0.2 degrees 2-theta; and combinations of these data.

From H2 of Capivasertib dihydrochloride salt may be further characterized by an X-ray powder diffraction pattern having peaks at 3.9, 7.9, 14.2, 16.0 and 23.8 degrees 2-theta±0.2 degrees 2-theta, and also having any one, two, three or four additional peaks selected from 12.7, 13.5, 17.7 and 22.7 degrees 2-theta±0.2 degrees 2-theta.

From H2 of Capivasertib dihydrochloride salt may be further characterized by an X-ray powder diffraction pattern having peaks at 3.9, 7.9, 12.7, 13.5, 14.2, 16.0, 17.7, 22.7 and 23.8 degrees 2-theta±0.2 degrees 2-theta.

According to any aspect or embodiment, crystalline Capivasertib dihydrochloride salt form H2 may be polymorphically pure. Additionally, or alternatively, the crystalline Capivasertib dihydrochloride salt form H2 may be enantiomerically pure.

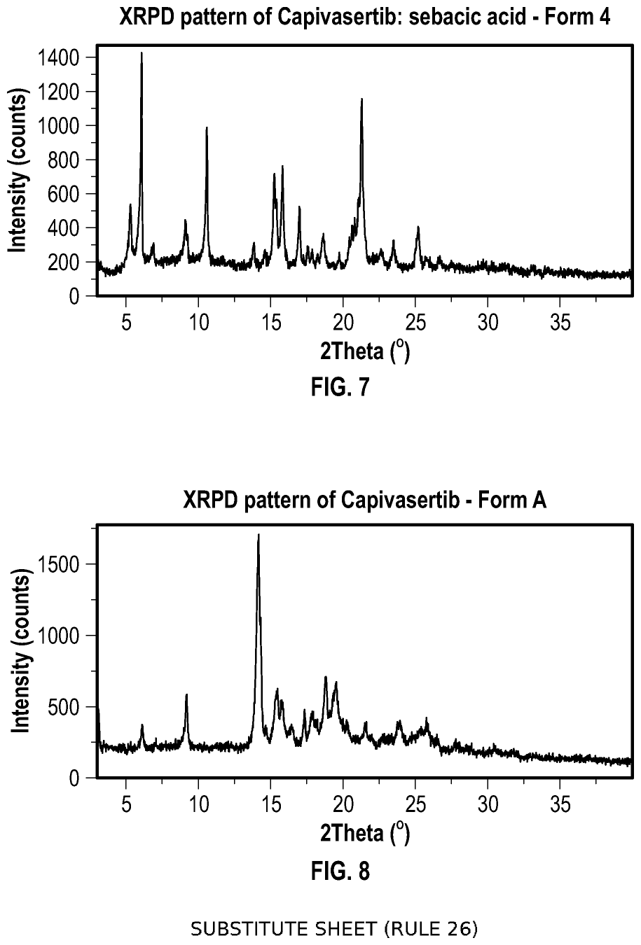

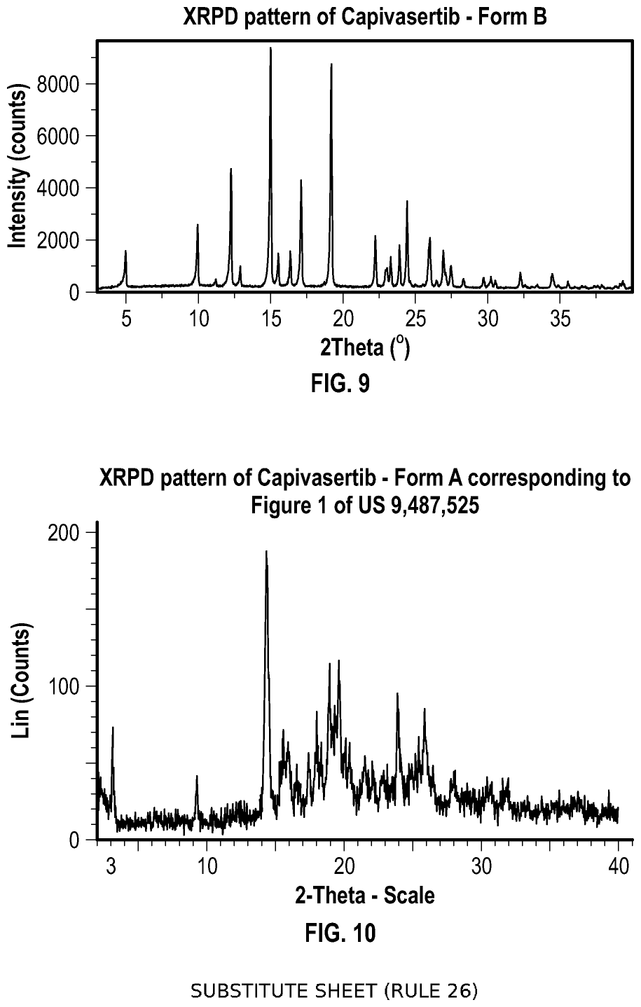

The present disclosure also includes a crystalline form of Capivasertib:sebacic acid, designated Form 4. Form 4 of Capivasertib:sebacic acid may be characterized by data selected from one or more of the following: an X-ray powder diffraction pattern substantially as depicted in FIG. 7; an X-ray powder diffraction pattern having peaks at 5.3, 6.1, 10.6, 18.6 and 21.3 degrees 2-theta±0.2 degrees 2-theta; and combinations of these data.

Form 4 of Capivasertib:sebacic acid may be further characterized by an X-ray powder diffraction pattern having peaks at 5.3, 6.1, 10.6, 18.6 and 21.3 degrees 2-theta±0.2 degrees 2-theta, and also having any one, two, three or four additional peaks selected from 6.9, 9.1, 15.8 and 25.2 degrees 2-theta±0.2 degrees 2-theta.

Form 4 of Capivasertib:sebacic acid may be further characterized by an X-ray powder diffraction pattern having peaks at 5.3, 6.1, 6.9, 9.1, 10.6, 15.8, 18.6, 21.3 and 25.2 degrees 2-theta±0.2 degrees 2-theta.

In embodiments of the present disclosure, crystalline Form 4 of Capivasertib:sebacic is isolated.

In one embodiment, crystalline Form 4 of Capivasertib:sebacic acid may be a co-crystal of Capivasertib and sebacic acid.

In another embodiment, crystalline Form 4 of Capivasertib:sebacic acid may be a salt.

In any embodiment, the ratio of Capivasertib to sebacic acid in Form 4 of Capivasertib:sebacic acid is 1:3 to 3:1, or 1.5:1 to 2.5:1, or 1.8:1 to 2.2:1, or 1.9:1 to 2.1, about 2:1. Particularly, the ratio Capivasertib:sebacic acid is 2:1.

According to any aspect or embodiment, crystalline Capivasertib:sebacic acid Form 4 may be polymorphically pure. Additionally, or alternatively, the crystalline Capivasertib:sebacic acid Form 4 may be enantiomerically pure.

According to any aspect or embodiment of the present disclosure, any of the disclosed crystalline forms of Capivasertib, Capivasertib salts, or Capivasertib co-crystals may be polymorphically pure or may be substantially free of any other solid state forms of the subject Capivasertib, Capivasertib salts, or Capivasertib co-crystals (for example crystalline form 1 of Capivasertib:methyl paraben, which is polymorphically pure, may be substantially free of any other solid state forms of Capivasertib:methyl paraben; crystalline form 2 Capivasertib:glutaric acid which is polymorphically pure, may be substantially free of any other solid state forms of Capivasertib:glutaric acid, and crystalline form 3 of Capivasertib: propyl paraben which is polymorphically pure, may be substantially free of any other solid state forms of Capivasertib: propyl paraben). In any aspect or embodiment of the present disclosure, any of the solid state forms of Capivasertib, Capivasertib salts, or Capivasertib co-crystals described in any aspect or embodiment disclosed herein, may contain: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of any other solid state forms of the subject Capivasertib, Capivasertib salt, or Capivasertib co-crystal, preferably as measured by XRPD. Thus, any of the disclosed crystalline forms of Capivasertib, Capivasertib salts, or Capivasertib co-crystals, described herein may be substantially free of any other solid state forms of the subject Capivasertib, Capivasertib salt, or Capivasertib co-crystal respectively, and may contain greater than about 80% (w/w), greater than about 90% (w/w), greater than about 95% (w/w), greater than about 98% (w/w), greater than about 99% (w/w), or about 100% of the subject solid state form of Capivasertib, Capivasertib salt, or Capivasertib co-crystal.

In any aspect or embodiment of the present disclosure, any of the solid state forms of Capivasertib, Capivasertib salts, or Capivasertib co-crystals described herein may be enantiomerically pure or may be substantially free of any enantiomeric forms of the subject Capivasertib. In particular, in any aspect or embodiment of the present disclosure, any of the solid state forms of Capivasertib, Capivasertib salts, or Capivasertib co-crystals may be enantiomerically pure or substantially free of other enantiomers of Capivasertib, i.e. in any aspect or embodiment of the present invention. Thus, a solid state form of Capivasertib, Capivasertib salt, or Capivasertib co-crystal according to any aspect or embodiment of the disclosure may be enantiomerically pure. Accordingly, the Capivasertib in Capivasertib:methyl paraben Form I, which is polymorphically pure, may be substantially free of other enantiomers (e.g. the 1R-isomer of Capivasertib). In any aspect or embodiment of the present disclosure, the Capivasertib in any of the solid state forms of Capivasertib, Capivasertib salts, or Capivasertib co-crystals, may contain: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of other enantiomers of Capivasertib, particularly the 1(R)-isomer of Capivasertib, preferably as measured by chiral HPLC. Thus, in any of the disclosed crystalline forms of Capivasertib, Capivasertib salts, or Capivasertib co-crystals, described herein, the Capivasertib may be substantially free of the 1R-isomer of Capivasertib, and preferably may contain greater than about 80% (w/w), greater than about 90% (w/w), greater than about 95% (w/w), greater than about 98% (w/w), greater than about 99% (w/w), greater than about 99.5% (w/w), greater than about 99.8% (w/w), greater than about 99.9% (w/w), or about 100% of the 1(S)-isomer.

The above crystalline polymorphs can be used to prepare other crystalline polymorphs of Capivasertib, Capivasertib salts, co-crystals and their solid state forms.

The present disclosure encompasses a process for preparing other solid state forms of Capivasertib, Capivasertib salts or co-crystals and solid state forms thereof. The process includes preparing any one of the crystalline polymorphs of Capivasertib, or a Capivasertib salt or a co-crystal by the processes of the present disclosure, and converting that crystalline polymorph, salt or co-crystal to said other crystalline polymorphs of Capivasertib, Capivasertib salts or co-crystals.

The present disclosure provides the above-described crystalline polymorphs of Capivasertib, Capivasertib salt or Capivasertib co-crystal for use in the preparation of pharmaceutical compositions comprising crystalline polymorphs, salts and co-crystals of Capivasertib thereof.

The present disclosure also encompasses the use of crystalline polymorphs of Capivasertib, Capivasertib salt or Capivasertib co-crystal of the present disclosure for the preparation of pharmaceutical compositions of crystalline polymorph Capivasertib or Capivasertib salt or Capivasertib co-crystal.

The present disclosure includes processes for preparing the above mentioned pharmaceutical compositions. The processes include combining any one or a combination of the crystalline polymorphs of Capivasertib or Capivasertib salt or Capivasertib co-crystal of the present disclosure with at least one pharmaceutically acceptable excipient.

Pharmaceutical combinations or formulations of the present disclosure contain any one or a combination of the solid state forms of Capivasertib or Capivasertib salt or Capivasertib co-crystal of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Capivasertib or Capivasertib salt or Capivasertib co-crystal and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Capivasertib or Capivasertib salt or Capivasertib co-crystal can be administered. Capivasertib, its salts or co-crystals may be formulated for administration to a mammal, in embodiments to a human, by injection. Capivasertib its salts or co-crystals can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Capivasertib, or Capivasertib salt or Capivasertib co-crystal and the pharmaceutical compositions and/or formulations of Capivasertib or Capivasertib salt or Capivasertib co-crystal of the present disclosure can be used as medicaments, in embodiments in the treatment of cancer, such as metastatic breast cancer.

The present disclosure also provides methods of treating cancer, such as breast cancer by administering a therapeutically effective amount of any one or a combination of the crystalline polymorphs of Capivasertib or Capivasertib salt or Capivasertib co-crystal of the present disclosure, or at least one of the above pharmaceutical compositions and/or formulations, to a subject in need of the treatment.

According to another aspect of the invention, there is provided a process for obtaining crystalline Form A of Capivasertib, which comprises combining a mixture of Capivasertib in 2-propanol with n-heptane. In any embodiment of this process, the mixture of Capivasertib and 2-propanol is heated to obtain a clear solution prior the addition of the anti-solvent.

According to a further aspect, there is provided a process for crystalline Form B of Capivasertib, which comprises combining a mixture of Capivasertib in acetone, dimethyl sulfoxide (DMSO); or mixtures thereof with water. In any embodiment of the process, the water may be cooled (preferably, below 10° C.; most preferably to about 5° C.) before combining it with a mixture of Capivasertib in the organic solvent(s). Alternatively, Form B can be obtained by a process, which comprises combining Capivasertib with a mixture of acetone:water. In a preferred embodiment, the mixture of Capivasertib in acetone:water is heated to obtain a clear solution and then cooled to room temperatures to initiate crystallization. In any embodiment of the process, the ratio (v/v) acetone:water is about 1:2 to about 2:1; about 1:1 to about 1:2; or about 1:2.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

Sample after being powdered in a mortar and pestle is applied directly on a silicon plate holder. The X-ray powder diffraction pattern was measured with Philips X'Pert PRO X-ray powder diffractometer, equipped with Cu irradiation source=1.54184 Å (Ångstrom), X'Celerator (2.022° 20) detector. Scanning parameters: angle range: 3-40 deg., step size 0.0167, time per step 37 s, continuous scan.

### EXAMPLES

### Preparation of Starting Materials

The starting material, Capivasertib (Form B) can be prepared according to methods known from the literature, for example U.S. Pat. No. 9,487,525.

### Example 1: Preparation of Crystalline Capivasertib: Methyl Paraben; Form 1

Capivasertib (148 mg) and methyl paraben (52 mg, 1 eq.) were suspended in 2 ml of water at 40° C. Suspension was vacuum filtered after 24 hours and analyzed by XRPD. Form 1 of Capivasertib:methylparaben was obtained; as shown in FIG. 1. 1:1 ratio (Capivasertib:methyl paraben) was confirmed by HPLC analysis.

### Example 2: Preparation of Crystalline Capivasertib:Glutaric Acid; Form 2

Capivasertib (152 mg) and glutaric acid (48 mg, 1 eq.) were suspended in 2 ml of acetone at 40° C. Suspension was vacuum filtered after 24 hours and analyzed by XRPD. Form 2 of Capivasertib:glutaric acid was obtained; as shown in FIG. 2. 1:1 ratio (Capivasertib:glutaric acid) was confirmed by HPLC analysis.

### Example 3: Preparation of Crystalline Capivasertib:Propyl Paraben; Form 3

Capivasertib (108 mg) and propyl paraben (92 mg, 2 eq.) were suspended in 2 ml of acetone at 40° C. Suspension was vacuum filtered after 24 hours and analyzed by XRPD. Form 3 of Capivasertib: propyl paraben was obtained; as shown in FIG. 3.

### Example 4: Preparation of Capivasertib Hydrochloride Salt; Form H1

Capivasertib (250 mg) was dissolved in 8 ml of 2-propanol at 64° C. Solution was cooled to 40° C. and 1.2 ml HCl (1M solution in ethyl acetate) was added dropwise to the solution. Crystallization was occurred momentary. Suspension was stirred for 3 hours and vacuum filtered. Form H1 of Capivasertib hydrochloride salt was obtained; as shown in FIG. 4.

### Example 5: Preparation of Capivasertib Dihydrochloride Salt; Form 112

Capivasertib (250 mg) was dissolved in 7 ml of 2-propanol at 64° C. Solution was cooled to 40° C. and 1.5 ml of HCl (5 M solution in 2-propanol) was added dropwise to the solution. Crystallization was occurred momentary. Suspension was stirred for 3 hours and vacuum filtered. Form H2 of Capivasertib dihydrochloride salt was obtained; as shown in FIG. 5.

### Example 6: Preparation of Capivasertib Amorphous

Capivasertib (500 mg) was dissolved in 7 ml of methanol at 38° C. Fast evaporation of solvent was performed at rotary evaporator (50° C./0 mbar). The obtained solid was analyzed by XRPD-amorphous Capivasertib; as shown in FIG. 6.

### Example 7: Preparation of Crystalline Capivasertib: Sebacic Acid; Form 4

Capivasertib (136 mg) and sebacic acid (64 mg; 1 equivalent) were suspended in 2 ml of methyl isobutyl ketone at 40° C. After 24 hours, the suspension was vacuum filtered and additionally suspended in a mixture of (1:1) methyl isobutyl ketone: 2-propanol (2 ml) at 40° C. Suspension was vacuum filtered after 24 hours and analyzed by XRPD. Form 4 of Capivasertib:sebacic acid was obtained, as shown in FIG. 7. 2:1 ratio (Capivasertib:sebacic acid) was confirmed by HPLC analysis.

### Example 8: Preparation of Crystalline Capivasertib—Form A

Capivasertib (100 mg) was dissolved in 2-propanol (3 ml) at 55° C. Solution was slowly cooled to room temperature (stirring at 350 rpm) and n-heptane (9 ml) was added dropwise. The obtained suspension was stirred for 2 hours (350 rpm), vacuum filtered at room temperature and vacuum dried at 80° C. for 2 hours.

Material was analyzed by XRFD-Capivasertib Form B was obtained; as shown in FIG. 8.

### Example 9: Preparation of Crystalline Capivasertib—Form B

Capivasertib (100 mg) was dissolved in a mixture of acetone:water (1:2 (v/v); 3 ml) at 57° C. Solution was cooled to room temperature and stirred (325 rpm) for 2 hours; vacuum filtered at room temperature and vacuum dried at 80° C. for 2 hours. Material was analyzed by XRFD-Capivasertib Form B was obtained.

### Example 10: Preparation of Crystalline Capivasertib—Form B

Capivasertib (100 mg) was dissolved in a mixture of acetone:DMSO (2:1 (v/v); 1.5 ml) at room temperature. 4.5 ml of cooled water (about 5° C.) were added dropwise. The obtained suspension was stirred for 2 hour (320 rpm), vacuum filtered at room temperature and vacuum dried at 80° C. for 2 hours. Material was analyzed by XRFD-Capivasertib Form B was obtained.

### Example 11: Preparation of Crystalline Capivasertib—Form B

Capivasertib (100 mg) was dissolved in DMSO (1.5 ml) at room temperature. 4.5 ml of cooled water (about 5° C.) were added dropwise. The obtained suspension was stirred for 2 hours (300 rpm); vacuum filtered at room temperature and vacuum dried at 80° C. for 2 hours. Material was analyzed by XRFD-Capivasertib Form B was obtained.

1. A co-crystal of Capivasertib with an alkyl ester of para-hydroxybenzoic acid.

2. A co-crystal of Capivasertib according to claim 1 which is a C₁ to C₆ alkyl ester of para-hydroxybenzoic, a C₁ to C₃ alkyl ester of para hydroxybenzoic para-hydroxybenzoic acid, or methyl ester of para-hydroxybenzoic acid.

3. A co-crystal of Capivasertib according to claim 1 which is Capivasertib:methyl paraben.

4. A co-crystal of Capivasertib:methyl paraben according to claim 3, designated as Form 1, and characterized by data selected from one or more of the following:
a. an XRPD pattern having peaks at 3.6, 7.2, 10.8, 11.7 and 13.4 degrees two theta±0.2 degrees two theta;
b. an XRPD pattern as depicted in FIG. 1;
c. an XRPD pattern having peaks at 3.6, 7.2, 10.8, 11.7 and 13.4 degrees two theta±0.2 degrees two theta, and also having one, two, three, or four additional peaks selected from 15.7, 17.9, 19.3 and 19.9 degrees two theta±0.2 degrees two theta;
and combinations of any a to c.

5. A crystalline salt of Capivasertib selected from Capivasertib glutaric acid salt, Capivasertib sebacic acid salt, Capivasertib hydrochloride salt and Capivasertib dihydrochloride salt.

6. A crystalline Capivasertib glutaric acid salt according to claim 5, designated as Form 2, and characterized by data selected from one or more of the following:
a. an XRPD pattern having peaks at 7.0, 10.6, 15.9, 17.9 and 20.9 degrees two theta±0.2 degrees two theta;
b. an XRPD pattern as depicted in FIG. 2;
c. an XRPD pattern having peaks at 7.0, 10.6, 15.9, 17.9 and 20.9 degrees two theta±0.2 degrees two theta, and also having one, two, three, four, or five additional peaks selected from 5.3, 9.6, 15.3, 18.4 and 24.9 degrees two theta±0.2 degrees two theta;
and combinations of any a to c.

7. A crystalline Capivasertib sebacic acid salt according to claim 5, designated as Form 4, and characterized by data selected from one or more of the following:
a. an XRPD pattern having peaks at 5.3, 6.1, 10.6, 18.6 and 21.3 degrees two theta±0.2 degrees two theta;
b. an XRPD pattern as depicted in FIG. 7;
c. an XRPD pattern having peaks at 5.3, 6.1, 10.6, 18.6 and 21.3 degrees two theta±0.2 degrees two theta, and also having one, two, three, or four additional peaks selected from 6.9, 9.1, 15.8 and 25.2 degrees two theta±0.2 degrees two theta;
and combinations of any a to c.

8. A crystalline Capivasertib hydrochloric acid salt according to claim 5, designated as Form H1, and characterized by data selected from one or more of the following:
a. an XRPD pattern having peaks at 11.6, 13.5, 17.4, 22.5 and 23.7 degrees two theta±0.2 degrees two theta;
b. an XRPD pattern as depicted in FIG. 4;
c. an XRPD pattern having peaks at 11.6, 13.5, 17.4, 22.5 and 23.7 degrees two theta±0.2 degrees two theta, and also having one, two, three, or four additional peaks selected from 12.7, 15.3, 16.1, 18.2 and 27.8 degrees two theta±0.2 degrees two theta;
and combinations of any a to c.

9. A crystalline Capivasertib dihydrochloric acid salt according to claim 5, designated as Form H2, and characterized by data selected from one or more of the following:
a. an XRPD pattern having peaks at 3.9, 7.9, 14.2, 16.0 and 23.8 degrees two theta±0.2 degrees two theta;
b. an XRPD pattern as depicted in FIG. 5;
c. an MOD pattern having peaks at 3.9, 7.9, 14.2, 16.0 and 23.8 degrees two theta±0.2 degrees two theta, and also having one, two, three, or four additional peaks selected from 12.7, 13.5, 17.7 and 22.7 degrees two theta±0.2 degrees two theta;
and combinations of any a to c.

10. A crystalline Capivasertib product according to claim 1, which is polymorphically pure, or which is substantially free of any other solid state forms of the Capivasertib product.

11. A crystalline Capivasertib product according to claim 1, which is enantiomerically pure, or which is substantially free of any enantiomers of Capivasertib.

12. A pharmaceutical composition comprising a crystalline product according to claim 1, and at least one pharmaceutically acceptable excipient.

13. (canceled)

14. A process for preparing a pharmaceutical composition, comprising combining the crystalline product according to claim 1 with at least one pharmaceutically acceptable excipient.

15. A medicament comprising the crystalline product according to claim 1.

16. (canceled)

17. A method of treating cancer comprising administering a therapeutically effective amount of crystalline product according to claim 1 to a subject in need of the treatment.
